(19) United States
(12) Patent Application Publication
(10) Pub. No.: US 2012/0030837 A1
(43) Pub. Date: Feb. 2, 2012

(54) MISCANTHUS TRANSFORMATION METHODS

(76) Inventor: Anthony Thinh Ngoc Trieu, Oxnard, CA (US)

(21) Appl. No.: 13/131,968

(22) PCT Filed: Dec. 1, 2009
(86) PCT No.: PCT/US09/66241

§ 371 Date: Jul. 27, 2011

Related U.S. Application Data
(60) Provisional application No. 61/118,790, filed on Dec. 1, 2008.

(51) Int. Cl. A01H 5/00; C12N 15/82

(52) U.S. Cl. 800/294; 435/469; 800/320

(57) ABSTRACT

Methods and materials for genetic transformation of Miscanthus in tissue culture via Agrobacterium are described.

### CROSS-REFERENCE TO RELATED APPLICATIONS

This application claims the benefit of U.S. Provisional Application Ser. No. 61/118,790, filed Dec. 1, 2008. The disclosure of the prior application is incorporated by reference in its entirety.

### TECHNICAL FIELD

This document relates to methods and materials involved in genetic transformation of Miscanthus plants. For example, this document provides methods for transforming Miscanthus plants via Agrobacterium-mediated transformation as well as transformed Miscanthus plants.

### BACKGROUND

There is a need for improved transformation procedures for Miscanthus. For example, particle bombardment-mediated transformation has achieved unsatisfactory results due to the complexity of screening and assaying transgenic Miscanthus calli in comparison to rice (Zili et al., High Technology Letters 10(3):27-31 (2004)). There is a need for methods of genetic transformation of Miscanthus plants that provide stability and efficiency. There is also a need for methods of genetic transformation of Miscanthus plants that are applicable to multiple clones, varieties, and/or species of Miscanthus.

### SUMMARY

This document relates to methods and materials involved in genetic transformation of Miscanthus plants. For example, methods for transforming Miscanthus plants via Agrobacterium-mediated transformation as well as transformed Miscanthus plants are provided herein. Methods and materials described herein can provide efficient transformation of Miscanthus calli, and expand the range of Miscanthus genotypes that can be transformed.

In general, one aspect of this document provides a method of transforming Miscanthus. The method comprises (a) contacting an embryogenic Miscanthus callus with Agrobacterium comprising an exogenous nucleic acid, which comprises a nucleotide sequence encoding a selectable marker conferring resistance to a selection agent, in a medium comprising 1/10 MS basal medium, 1/10 B5 vitamins, 3% maltose, 200 mg/L L-cysteine, 2 mg/L 2,4-D, 0.5 mg/L kinetin, and at least 300 μM acetosyringone, at pH 5.4 under conditions wherein the callus is infected by the Agrobacterium, and (b) selecting for at least one transformed callus, derived from step (a), in a medium that contains the selection agent, and inhibits the growth of the Agrobacterium. The selectable marker can confer herbicide resistance. The selectable marker can be a phosphinothricin acetyltransferase polypeptide, a glyphosate resistant 3-enolpyruvylshikimate-5-phosphate synthate polypeptide or a glyphosate oxidoreducatase polypeptide. The selectable marker can confer antibiotic resistance. The selectable marker can be a neomycin phosphotransferase II polypeptide. The exogenous nucleic acid can further comprise a nucleotide sequence encoding a screenable marker. The screenable marker can be a GFP polypeptide or a β-glucuronidase polypeptide.

The method can further comprise culturing the Agrobacterium-infected callus on the medium for 5-7 days. The method can further comprise culturing the callus derived from step (a) on a recovery medium comprising N6 basal medium, 0.8 mg/L KI, 0.025 mg/L CoCI₂.6H₂O, 0.025 mg/L CuSO₄.5H₂O, 0.25 mg/L NaMoO₄.2H₂O, 2 mg/L glycine, 100 mg/L myo-inositol, 5 mg/L thiamine HCl, 1 mg/L pyridoxine HCl, 1 mg/L nicotinic acid, 1 g/L casamino acid, 2 mg/L 2,4-D, 1.0 mg/L BA, 3% maltose, 125 mg/L TIMENTIN®, and 7.0 g/L agar at pH 5.8 for 5-7 days. The recovery medium further can include 1 g/L asparagine and 2 g/L proline.

The method can further comprise inducing the embryogenic callus from Miscanthus tissue on an induction medium prior to the contacting step. The Miscanthus tissue can be an immature inflorescence, a germinated seed, or a shoot apex. The Miscanthus tissue can be an explant from a Miscanthus plant selected from the group consisting of Miscanthus sinensis, Miscanthus x giganteus, Miscanthus floridulus and Miscanthus sacchariflorus. The inducing step can comprise subculturing primary calli on the induction medium every two weeks until the embryogenic callus is formed. The induction medium can comprise 2,4-D and kinetin in a four to one ratio. The induction medium can comprises N6 basal medium, 0.8 mg/L KI, 0.025 mg/L CoCI₂.6H₂O, 0.025 mg/L CuSO₄.5H₂O, 0.25 mg/L NaMoO₄.2H₂O, 2 mg/L glycine, 100 mg/L myo-inositol, 5 mg/L thiamine HCl, 1 mg/L pyridoxine HCl, 1 mg/L nicotinic acid, 1 g/L casamino acid, 2 mg/L 2,4-D, 0.5 mg/L kinetin, 3% maltose, and 3.5 g/L GELRITE®, at pH 5.8. The method can further comprise regenerating at least one Miscanthus plant from the transformed callus. The regeneration step can comprise culturing the transformed callus on a medium comprising MS basal salts, B5 vitamins, 2 mg/L BA, 0.1 mg/L NAA, 1% maltose, 50 mg/L L-glutamine, 2% sucrose, 7 g/L agar, 50 mg/L paromomycin, and 125 mg/L TIMENTIN®, at pH 5.8. The regeneration step can comprise subsequently culturing the transformed callus on a medium comprising ½ MS basal salts, 0.2 mg/L glycine, 0.5 mg/L thiamine, 1.0 mg/L pyridoxine, 1.0 mg/L nicotinic acid, 100 mg/L myo-inositol, 1.5% sucrose, 0.5 mg/L NAA, 125 mg/L TIMENTIN®, and 2.5 g/L GELRITE®, at pH 5.8.

In another aspect, this document provides a transgenic Miscanthus plant. The plant can comprise an exogenous nucleic acid comprising a selectable marker and at least one T-DNA border. The T-DNA border can be a right T-DNA border.

This document also features a method of transforming Miscanthus. The method includes (a) contacting an embryogenic Miscanthus callus with Agrobacterium comprising an exogenous nucleic acid, wherein the exogenous nucleic acid includes a nucleic acid sequence encoding a selectable marker conferring resistance to a selection agent; and (b) selecting for at least one transformed callus, derived from step (a), in a medium containing the selection agent, wherein the selection medium inhibits the growth of said Agrobacterium.

In yet another aspect, this document features a method of transforming Miscanthus. The method includes (a) inducing an embryogenic callus from Miscanthus tissue on an induction medium; (b) contacting the induced embryogenic callus with Agrobacterium comprising an exogenous nucleic acid, wherein the exogenous nucleic acid includes a nucleic acid sequence encoding a selectable marker conferring resistance to a selection agent; (c) selecting for at least one transformed callus, derived from step (b), in a medium containing the selection agent, wherein the selection medium inhibits the growth of the Agrobacterium; and (d) regenerating at least one Miscanthus plant from the transformed callus.

This document also features a transgenic Miscanthus plant. The plant includes an exogenous nucleic acid comprising at least one T-DNA border incorporated into its genome.

Unless otherwise defined, all technical and scientific terms used herein have the same meaning as commonly understood by one of ordinary skill in the art to which this invention belongs. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description.

### DESCRIPTION OF THE DRAWINGS

FIG. 1. GFP expressing callus of Miscanthus sinensis on selection media, 60 days after infection with Agrobacterium containing an NB4Kan:p326-EGFP construct viewed under UV light (left) and visible light (right).

FIG. 2. GFP expressing callus (right), and non-GFP expressing callus (left) of Miscanthus x giganteus clone ‘Illinois’ on selection media, 75 days after infection with Agrobacterium containing a NB4Kan:p326-EGFP construct.

FIG. 3. GFP-expressing callus of Miscanthus sacchariflorus ‘Robusta’ on selection media, 60 days after infection with Agrobacterium containing an NB4Kan:p326-EGFP construct. (Upper panels). GFP-expressing shoots, 10 days after transfer to regeneration medium with 50 mg/L paromomycin. (Lower panels).

FIG. 4. Map of shuttle vector NB4KanEGFP. “Spec” denotes a nucleic acid sequence encoding a spectinomycin adenyltransferase polypeptide. TrfA and OriV are from plasmid RK2. On is from plasmid pBR322. Right border (RB) and Left border (LB) are from Agrobacterium tumefaciens Ti plasmid. 35S is a promoter from cauliflower mosaic virus. NPT II sequence encodes a neomycin phosphotransferase polypeptide. “NOS” is a terminator of the NOS gene of Agrobacterium. Promoter 35S and NOS terminator regulate RNA transcription of plant selection marker NPT II. 326FL is a plant promoter from Arabidopsis, EGFP is enhanced green fluorescence protein. 3′ OCS is terminator of OCS gene from Agrobacterium. Promoter 326FL and 3′ OCS terminator regulate RNA transcription of EGFP.

### DETAILED DESCRIPTION

This document relates to methods and materials involved in genetic transformation of Miscanthus plants. For example, methods for transforming Miscanthus plants via Agrobacterium-mediated transformation as well as transformed Miscanthus plants and products of transgenic Miscanthus plants, are provided herein. For example, media for Miscanthus callus induction, co-cultivation of Miscanthus calli and Agrobacterium, recovery of co-cultured calli, selection of transformed calli, regeneration of transformed calli, and rooting of transgenic Miscanthus plants, are provided herein.

### I. MISCANTHUS TRANSFORMATION

In general, transformation of Miscanthus comprises Agrobacterium-mediated plant transformation and plant propagation via somatic embryogenesis. The methods described herein can comprise several stages. For example, transformation can include inducing embryogenic calli from plant tissue, preparing Agrobacterium transformants, infecting embryogenic calli with prepared Agrobacterium, selecting transformed calli, regenerating a plant from a transformed callus, and rooting the regenerated plant. Each stage is described in detail below.

The methods described herein can use solid medium or liquid medium. A solid medium can be, for example, Murashige and Skoog (MS) medium containing agar and a suitable concentration of an auxin, such as 2,4-dichlorophenoxyacetic acid (2,4-D), and a suitable concentration of a cytokinin, such as kinetin. In some cases, when using solid medium, plant tissue can be placed directly onto the medium or can be placed onto a filter that is then placed in contact with the medium. When liquid medium is used, plant tissue can be placed onto a flotation device, such as a porous membrane that contacts the liquid medium.

The medium compositions described herein can include an auxin, a cytokinin, or a combination thereof, for plant propagation via somatic embryogenesis. Suitable cytokinins can include 6-Benzyl aminopurine (or benzyladenine (BA)), kinetin, zeatin, adenosine phosphate, thidiazuron (TDZ) and other cytokinin-like compounds. Suitable auxins can include indole-3-acetic acid (IAA), 2,4-D, 1-naphthaleneacetic acid (NAA), indole-3-butryic acid (IBA), 2-methoxy-3,6-dichlorobenzoic acid (dicamba), and other auxin-like compounds.

In some embodiments, the methods described herein can provide transformed Miscanthus in about 8, 9, 10, 11, 12, 13, 14, or 16 weeks. In some cases, collection of tissue, callus induction, infection, selection, and regeneration of transformed Miscanthus plants can be completed within about 8, 9, 10, 11, 12, 13, 14, or 16 weeks.

### A. Callus Induction

Embryogenic calli can be used to produce a transformed Miscanthus plant. Plant tissue can be prepared by suitable techniques for somatic embryogenesis. For example, a plant tissue (explant) can be harvested from in vitro, greenhouse, or field-grown plants. Appropriate explant types can include, shoot apices, immature inflorescences, immature and mature embryos, leaves, and roots, for example. An explant can be harvested from including a newly germinated Miscanthus plant or a shoot culture. In some cases, an explant can be dissected into about 1 mm to about 1 cm sections.

The prepared explant can be cultured in a callus induction media to form plant calli and embryogenic calli. In some embodiments, a callus induction media can comprise a basal medium (e.g., N6, MS, NB, and Gamborg B-5); nutrients (e.g., potassium (K) or potassium iodide (KI)); micronutrients (e.g., cobalt chloride (CoCI₂.6H₂O), cupric sulfate (CuSO₄.5H₂O), and sodium molybdate (NaMoO₄.2H₂O)); amino acids (e.g., glycine and casamino acid); vitamins (e.g., myo-inositol, thiamine HCl, pyridoxine HCl, and nicotinic acid); auxin (e.g., 2,4-D); cytokinin (e.g., kinetin and BA); sugar (e.g., sucrose or maltose); and a gelling agent (e.g., agar or GELRITE®). In some embodiments, an induction medium can have a neutral to acidic pH. In some embodiments, an induction media can comprise N6 basal medium, about 0.20 to 10.00 mg/L KI, about 0.0125 to 0.10 mg/L CoCI₂.6H₂O, about 0.0125 to 2.0 mg/L CuSO₄.5H₂O, about 0.05 to 1.00 mg/L NaMoO₄.2H₂O, about 0.50 to 10.00 mg/L glycine, about 10.00 to 500.00 mg/L myo-inositol, about 0.10 to 10.00 mg/L thiamine HCl, about 0.50 to 5.00 mg/L pyridoxine HCl, about 0.50 to 5.00 mg/L nicotinic acid, about 200 to 4,000 mg/L casamino acid, about 0.20 to 5.00 mg/L 2,4-D, about 0.10 to 2.00 mg/L kinetin, about 1 to 5% maltose, and about 2 to 4 g/L GELRITE®, at pH range of about 5.2 to 6.0. In some cases, the ratio of 2,4-D:BA can be about 8:1, 7:1, 6:1, 5:1, or 4:1. In some embodiments, an induction media can comprise N6 basal medium, about 0.8 mg/L KI, about 0.025 mg/L CoCI₂.6H₂O, about 0.025 mg/L CuSO₄.5H₂O, about 0.25 mg/L NaMoO₄.2H₂O, about 2.00 mg/L glycine, about 100 mg/L myo-inositol, about 5.00 mg/L thiamine HCl, about 1.00 mg/L pyridoxine HCl, about 1.00 mg/L nicotinic acid, about 1 g/L casamino acid, about 2 mg/L 2,4-D, about 0.5 mg/L kinetin, about 3% maltose, and about 3.5 g/L GELRITE®, at about pH 5.8.

Callus induction can occur within a range of tissue culture conditions. For example, the period of time that the plant tissue is cultured in induction media, the number of days until calli form, the temperature for incubation of the plant tissue, the photosynthetically active radiation (PAR), and the photoperiod, can vary. In some cases, callus induction can include culturing prepared tissue for about 2, 3, 4, or 5 weeks. In some cases, embryogenic calli can form in about 20, 21, 22, 23, 24, 25, 26, 27, 28, 29, or 30 days. In some cases, callus induction can include incubating prepared tissue at about 22, 23, 24, 25, 26, 27, or 28° C. In some cases, a PAR can have a photosynthetic photon flux density (PPFD) of 0 to 5 μmol/m²/s. In some cases, an appropriate photoperiod for calli induction can be about 10, 14, 16, 18, 20, 22, or 24 hours of light per day.

In some cases, embryogenic calli can be subcultured about every 1, 2, 3, 4, or 5 weeks on regeneration media, to determine the regeneration capacity of induced calli. For example, some induced calli can be transferred to a regeneration media in a petri dish, and monitored for formation of shoots, roots, and plantlets. The regeneration capacity can be estimated by counting the number of regenerated plantlets per petri dish, for example.

### B. Agrobacterium Preparation

Transforming Bacteria Cells

A number of Agrobacterium are suitable for use with the methods described herein. For example, Agrobacterium tumefaciens (Rhizobium radiobactor) strain C58, LBA4404, EHA101, EHA105, or EHA109 can be used to produce a transformed Miscanthus plant. In some cases, a strain of Agrobacterium rhizogenes (Rhizobium rhizogenes) can be used.

A recombinant nucleic acid construct with a marker for selection of transformed Agrobacterium can be introduced to competent Agrobacterium by a suitable bacterial transformation technique (see e.g., Hellens et al., “Technical Focus: a guide to Agrobacterium binary Ti vectors.” Trends Plant Sci. 5(10):446-451 (2000)). For example, a recombinant nucleic acid construct can be transferred into Agrobacterium through an electroporation process. Selection of transformed Agrobacterium can include inoculating a liquid medium containing a selective agent, such as an antibiotic, and allowing the bacterium to grow to an appropriate cell density. The cells can be transferred to a co-cultivation medium for infecting and transforming plant cells or tissue.

Exogenous Nucleic Acids

This document provides methods and materials relating to exogenous nucleic acid molecules and cells that contain an exogenous nucleic acid. The term “nucleic acid” as used herein encompasses both RNA and DNA, including cDNA, genomic DNA, and synthetic (e.g., chemically synthesized) DNA. The nucleic acid can be double-stranded or single-stranded. Where single-stranded, the nucleic acid can be the sense strand or the antisense strand. In addition, nucleic acid can be circular or linear.

An exogenous nucleic acid can be a naturally-occurring nucleic acid that is not immediately contiguous with both of the sequences with which it is immediately contiguous (one on the 5′ end and one on the 3′ end) in the naturally-occurring genome of the organism from which it is derived. For example, an exogenous nucleic acid molecule can be an isolated DNA molecule that is incorporated into a vector, an autonomously replicating plasmid, or into the genomic DNA of a prokaryote or eukaryote. In addition, an exogenous nucleic acid can include a nucleic acid molecule that is part of a hybrid or fusion nucleic acid sequence.

An exogenous nucleic acid can also include suitable non-naturally-occurring nucleic acid. For example, engineered nucleic acids can be made using common molecular cloning or chemical nucleic acid synthesis techniques. Isolated non-naturally-occurring nucleic acid can be independent of other sequences, or incorporated into a vector, an autonomously replicating plasmid, or the genomic DNA of a prokaryote or eukaryote. In addition, a non-naturally-occurring nucleic acid can include a nucleic acid molecule that is part of a hybrid or fusion nucleic acid sequence. An exogenous nucleic acid can include sequences encoding polypeptides, or transcription factors originating from various species, including, but not limited to, plants, algae, fungi, monera, bacteria, archaeobacteria, protista, and animals.

Agrobacterium can contain an exogenous nucleic acid for transfer to a Miscanthus cell or tissue. Agrobacterium are capable of transferring exogenous nucleic acids into plant cells, where the exogenous nucleic acids become stably integrated into genomic DNA. Typically, an exogenous nucleic acid integrated into a plant genome is flanked by one or both of the right and/or left T-DNA border sequences, 25-base-pair sequences of imperfect direct repeats that are required for transfer from Agrobacterium to a plant genome. A number of suitable nucleic acids can be included in an exogenous nucleic acid for use with the methods described herein. For example, an exogenous nucleic acid can be inserted in a recombinant nucleic acid construct, between a right T-DNA border sequence and a left T-DNA border sequence, for Agrobacterium-mediated transfer to a plant cell, using standard molecular biological techniques. In some cases, a recombinant nucleic acid construct for use with the methods described herein can include functional plant analogs of the Agrobacterium T-DNA borders. For example, plant transfer DNA sequences have been described in Rommens et al. (“Crop Improvement through Modification of the Plant's Own Genome,” Plant Physiology, 135: 421-431, 2004).

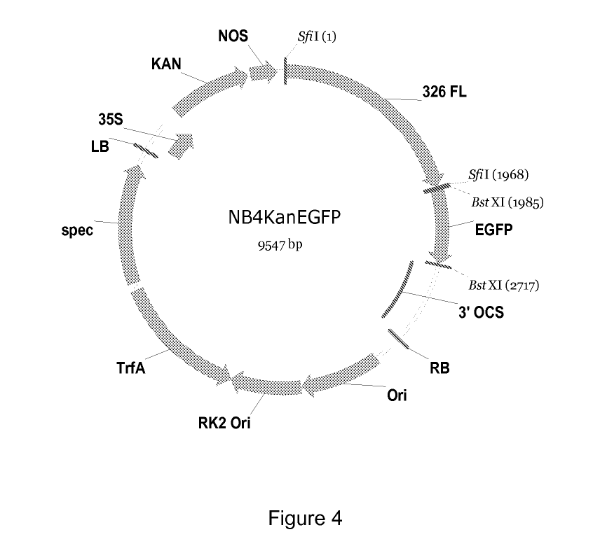

Exogenous nucleic acids for use with the methods described herein can include nucleotide sequences for regulating and reporting expression of the exogenous nucleic acid. For example, an appropriate exogenous nucleic acid can be a recombinant nucleic acid construct including origins of replication, scaffold attachment regions (SARs), and/or markers. For example, a recombinant nucleic acid construct can include, a marker for selection of Agrobacterium transformants, and a T-DNA region, comprising a polynucleotide (e.g., an NB4KanEGFP plasmid (FIG. 4)). In some cases, a recombinant nucleic construct can be a shuttle vector of a binary vector system or a superbinary vector (e.g., pBin19, pBI121, pCAMBIA series, pPZP series, pGreen series, pGA482, pSB11e, pSB1e, pPCV001, pCLD04541, pBIBAC series, and pYLTAC series as described in Komori et al., “Current Status of Binary Vectors and Superbinary Vectors,” Plant Physiology 145:1155-1160 (2007)).

An exogenous nucleic acid can include a nucleotide sequence encoding a screenable marker for conferring a screenable phenotype on a transformed plant cell. For example, a screenable marker can be used to detect the presence of a transferred exogenous nucleic acid, or measure the expression level of a transferred exogenous nucleic acid in a transformed plant cell or tissue. A screenable marker can be used with the methods described herein to assay transient expression. For example, appropriate screenable markers can include a beta-glucuronidase (GUS) polypeptide, a luciferase polypeptide, and a Green Fluorescent Protein (GFP) polypeptide. In addition, an expression vector can include a tag sequence designed to facilitate manipulation or detection (e.g., purification or localization) of the expressed polypeptide. Tag sequences, such as green fluorescent protein (GFP), glutathione S-transferase (GST), polyhistidine, c-myc, hemagglutinin, or Flag™ tag (Kodak, New Haven, Conn.) sequences typically are expressed as a fusion with the encoded polypeptide. Such tags can be inserted anywhere within the polypeptide, including at either the carboxyl or amino terminus.

At least one of the exogenous nucleic acids with which Miscanthus is transformed comprises a nucleotide sequence that, when expressed, confers a selectable phenotype on a plant cell. Expression of a selectable marker allows for preferential selection of stably transformed cells, tissues and/or plants, in the presence of a selection agent. In some cases, a selectable marker confers biocide resistance, such as resistance to an antibiotic (e.g., kanamycin, paromomycin, or hygromycin), an anti-neoplastic agent (e.g., methotrexate), or an herbicide (e.g., bromoxynil, chlorosulfuron or phosphinothricin).

In some embodiments, a selectable marker is a polypeptide that confers herbicide resistance on plants expressing the polypeptide. Herbicide resistance can be herbicide tolerance, such as tolerance to glyphosate and bromoynil. Polypeptides conferring resistance to a herbicide that inhibits the growing point or meristem, such as an imidazolinone or a sulfonylurea can be suitable. Exemplary polypeptides in this category can be mutant acetolactate synthase (ALS) and acetohydroxy acid synthase (AHAS) enzymes as described, for example, in U.S. Pat. Nos. 5,767,366 and 5,928,937. U.S. Pat. Nos. 4,761,373 and 5,013,659 are directed to plants resistant to various imidazolinone or sulfonamide herbicides. U.S. Pat. No. 4,975,374 relates to plant cells and plants containing a gene encoding a mutant glutamine synthetase (GS) resistant to inhibition by herbicides that are known to inhibit GS, e.g. phosphinothricin and methionine sulfoximine. U.S. Pat. No. 5,162,602 discloses plants resistant to inhibition by cyclohexanedione and aryloxyphenoxypropanoic acid herbicides. The resistance is conferred by an altered acetyl coenzyme A carboxylase (ACCase).

Polypeptides for resistance to glyphosate (sold under the trade name Roundup®) are also suitable. See, for example, U.S. Pat. No. 4,940,835 and U.S. Pat. No. 4,769,061. Typically, herbicide resistance is conferred by an altered 5-enolpyruvyl-3-phosphoshikimate (EPSP) synthase. Such polypeptides can confer resistance to glyphosate herbicidal compositions, including without limitation glyphosate salts such as the trimethylsulphonium salt, the isopropylamine salt, the sodium salt, the potassium salt and the ammonium salt (see e.g., U.S. Pat. Nos. 6,451,735 and 6,451,732). Polypeptides for resistance to phosphono compounds such as glufosinate ammonium or phosphinothricin, and pyridinoxy or phenoxy propionic acids and cyclohexones are also suitable (see e.g., European application No. 0 242 246, U.S. Pat. Nos. 5,879,903, 5,276,268 and 5,561,236). A polypeptide can confer resistance to herbicides that inhibit photosynthesis, such as a triazine and a benzonitrile (nitrilase) (e.g., U.S. Pat. No. 4,810,648) or herbicides including 2,2-dichloropropionic acid, sethoxydim, haloxyfop, imidazolinone herbicides, sulfonylurea herbicides, triazolopyrimidine herbicides, s-triazine herbicides and bromoxynil. Also suitable is a herbicide-resistant polypeptide such as a protox enzyme (see e.g., U.S. Patent Application No. 20010016956, and U.S. Pat. No. 6,084,155).

Sequences of interest that can be used in the methods described herein include, but are not limited to, sequences encoding genes or fragments thereof that modulate cold tolerance, frost tolerance, heat tolerance, drought tolerance, water used efficiency, nitrogen use efficiency, pest resistance, biomass, chemical composition, plant architecture, and/or biofuel conversion properties. In particular, exemplary sequences are described in the following applications which are incorporated herein by reference in their entirety: US20080131581, US20080072340, US20070277269, US20070214517, US20070192907, US20070174936, US20070101460, US 20070094750, US20070083953, US20070061914, US20070039067, US20070006346, US20070006345, US20060294622, US20060195943, US20060168696, US20060150285, US20060143729, US20060134786, US20060112454, US20060057724, US20060010518, US20050229270, US20050223434, and US20030217388.

A nucleotide sequence encoding a polypeptide or transcription product that affects abiotic stress resistance, including, but not limited to flowering, seed development, enhancement of nitrogen utilization efficiency, altered nitrogen responsiveness, drought resistance or tolerance, cold resistance or tolerance, and salt resistance or tolerance, and increased yield under stress, can be introduced into Miscanthus by the methods described herein (see e.g., PCT Application WO 00/73475 (modulating water-use efficiency through alteration of malate); U.S. Pat. No. 5,892,009, U.S. Pat. No. 5,965,705, U.S. Pat. No. 5,929,305, U.S. Pat. No. 5,891,859, U.S. Pat. No. 6,417,428, U.S. Pat. No. 6,664,446, U.S. Pat. No. 6,706,866, U.S. Pat. No. 6,717,034, U.S. Pat. No. 6,801,104, and PCT Applications WO 2000/060089, WO 2001/026459, WO 2001/035725, WO 2001/034726, WO 2001/035727, WO 2001/036444, WO 2001/036597, WO 2001/036598, WO 2002/015675, WO 2002/017430, WO 2002/077185, WO 2002/079403, WO 2003/013227, WO 2003/013228, WO 2003/014327, WO 2004/031349, WO 2004/076638, WO 98/09521, and WO 99/38977 (mitigating negative effects of freezing, high salinity, and drought on plants, as well as conferring other positive effects on plant phenotype); US 2004/0148654 and WO 01/36596 (improving agronomic phenotypes such as increased yield and/or increased tolerance to abiotic stress); PCT Applications WO 2000/006341 and WO 04/090143, U.S. application Ser. No. 10/817,483 and U.S. Pat. No. 6,992,237 (modulating cytokinin expression to increase stress tolerance, such as drought tolerance, and/or increased yield); also see PCT Applications WO 02/02776, WO 2003/052063, JP2002281975, U.S. Pat. No. 6,084,153, WO 01/64898, U.S. Pat. Nos. 6,177,275 and 6,107,547 (enhancement of nitrogen utilization and altered nitrogen responsiveness); and see, US20040128719, US20030166197, PCT Application WO 2000/32761 (ethylene alteration); and see, US20040098764 or US20040078852 (plant transcription factors or transcriptional regulators of abiotic stress)). Other polypeptides and transcription factors that affect plant growth and agronomic traits such as yield, flowering, plant growth and/or plant structure, can be introduced or introgressed into Miscanthus plants (see e.g., PCT Application WO 97/49811 (LHY), WO 98/56918 (ESD4), WO 97/10339 and U.S. Pat. No. 6,573,430 (TFL), U.S. Pat. No. 6,713,663 (FT), WO 96/14414 (CON), WO 96/38560, WO 01/21822 (VRN1), WO 00/44918 (VRN2), WO 99/49064 (GI), WO 00/46358 (FRI), WO 97/29123, U.S. Pat. No. 6,794,560, U.S. Pat. No. 6,307,126 (GAI), WO 99/09174 (D8 and Rht), and WO 2004/076638 and WO 2004/031349 (transcription factors)).

An exogenous nucleic acid can include a nucleotide sequence that results in male-sterility, e.g., pollen is either not formed or is nonviable. Suitable male-sterility systems include cytoplasmic male sterility (CMS), nuclear male sterility, genetic male sterility, and molecular male sterility. For example, an exogenous nucleic acid can be a transgene that inhibits microsporogenesis and/or pollen formation. A number of different methods of conferring male sterility are available. For example, multiple mutant genes or expression of transgenes can be used to silence one or more nucleic acid sequences necessary for male fertility (see e.g., U.S. Pat. Nos. 4,654,465, 4,727,219, and 5,432,068, EPO Publication No. 329, 308 and PCT Application WO 90/08828). In some cases, infertility can be achieved using a recombinant nucleic acid construct comprising a sequence encoding a deacetylase polypeptide, under the control of a tapetum-specific promoter, with the application of N-acetyl-phosphinothricin (see e.g., PCT Application WO 01/29237), various stamen-specific promoters (see e.g., PCT Applications WO 92/13956 and WO 92/13957) or using a barnase/barstar transgene system (see e.g., Paul et al., Plant Mol. Biol. 19:611-622 (1992)). Additional examples of nuclear male and female sterility systems and genes are described in U.S. Pat. No. 5,859,341; U.S. Pat. No. 6,297,426; U.S. Pat. No. 5,478,369; U.S. Pat. No. 5,824,524; U.S. Pat. No. 5,850,014; and U.S. Pat. No. 6,265,640.

An exogenous nucleic acid can be a polynucleotide, which includes a nucleotide sequence encoding a polypeptide and regulatory sequences such as transcription promoters. As used herein, “promoter” includes reference to a region of DNA upstream from the start of transcription and involved in recognition and binding of RNA polymerase and other proteins to initiate transcription. A “plant promoter” is a promoter capable of initiating transcription in plant cells. Examples of promoters under developmental control include promoters that can initiate transcription in certain tissues, such as leaves, roots, seeds, fibers, xylem vessels, tracheids, or sclerenchyma. Such promoters are referred to as “tissue-preferred”. Promoters that initiate transcription only in a certain tissue are referred to as “tissue-specific”. A “cell-type” specific promoter primarily drives expression in certain cell types in one or more organs, for example, vascular cells in roots or leaves. An “inducible” promoter is a promoter that is under environmental control. Examples of environmental conditions that may effect transcription by inducible promoters include anaerobic conditions or the presence of light. Tissue-specific, tissue-preferred, cell type specific, and inducible promoters constitute the class of “non-constitutive” promoters. A “constitutive” promoter is a promoter that is active under most environmental conditions.

The choice of regulatory regions to be included in a recombinant construct depends upon several factors, including, but not limited to, efficiency, selectability, inducibility, desired expression level, and cell- or tissue-preferential expression. It is a routine matter for one of skill in the art to modulate the expression of a coding sequence by appropriately selecting and positioning regulatory regions relative to the coding sequence. Transcription of a nucleic acid can be modulated in a similar manner. Some suitable regulatory regions initiate transcription only, or predominantly, in certain cell types. Methods for identifying and characterizing regulatory regions in plant genomic DNA are known, including, for example, those described in the following references: Jordano et al., Plant Cell, 1:855-866 (1989); Bustos et al., Plant Cell, 1:839-854 (1989); Green et al., EMBO J., 7:4035-4044 (1988); Meier et al., Plant Cell, 3:309-316 (1991); and Zhang et al., Plant Physiology, 110:1069-1079 (1996). Examples of various regulatory regions are described in more detail in. It will be appreciated that a regulatory region may meet criteria for one classification based on its activity in one plant species, and yet meet criteria for a different classification based on its activity in another plant species. Examples of regulatory regions include broadly expressing promoters, root promoters, maturing endosperm promoters, ovary tissue promoters, embryo sac/early endosperm promoters, embryo promoters, photosynthetic tissue promoters, vascular tissue promoters, inducible promoters, basal promoters, or other regulatory regions.

Examples of various classes of regulatory regions are described below. Some of the regulatory regions indicated below as well as additional regulatory regions are described in more detail in U.S. Patent Application Ser. Nos. 60/505,689; 60/518,075; 60/544,771; 60/558,869; 60/583,691; 60/619,181; 60/637,140; 60/757,544; 60/776,307; 10/957,569; 11/058,689; 11/172,703; 11/208,308; 11/274,890; 60/583,609; 60/612,891; 11/097,589; 11/233,726; 11/408,791; 11/414,142; 10/950,321; 11/360,017; PCT/US05/011105; PCT/US05/23639; PCT/US05/034308; PCT/US05/034343; and PCT/US06/038236; PCT/US06/040572; PCT/US07/62762; and U.S. Patent Application Pub. Nos. US20080072340, US20080134357, US20080044898, US20070277269, US20070226830, US20070136839, US 20070124834, and US20060107346.

For example, the sequences of regulatory regions p326, YP0144, YP0190, p13879, YP0050, p32449, 21876, YP0158, YP0214, YP0380, PT0848, PT0633, YP0128, YP0275, PT0660, PT0683, PT0758, PT0613, PT0672, PT0688, PT0837, YP0092, PT0676, PT0708, YP0396, YP0007, YP0111, YP0103, YP0028, YP0121, YP0008, YP0039, YP0115, YP0119, YP0120, YP0374, YP0101, YP0102, YP0110, YP0117, YP0137, YP0285, YP0212, YP0097, YP0107, YP0088, YP0143, YP0156, PT0650, PT0695, PT0723, PT0838, PT0879, PT0740, PT0535, PT0668, PT0886, PT0585, YP0381, YP0337, PT0710, YP0356, YP0385, YP0384, YP0286, YP0377, PD1367, PT0863, PT0829, PT0665, PT0678, YP0086, YP0188, YP0263, PT0743 and YP0096 are set forth in the sequence listing of PCT/US06/040572; the sequence of regulatory region PT0625 is set forth in the sequence listing of PCT/US05/034343; the sequences of regulatory regions PT0623, YP0388, YP0087, YP0093, YP0108, YP0022 and YP0080 are set forth in the sequence listing of U.S. patent application Ser. No. 11/172,703; the sequence of regulatory region PR0924 is set forth in the sequence listing of PCT/US07/62762; and the sequences of regulatory regions p530c10, pOsFIE2-2, pOsMEA, pOsYp102, and pOsYp285 are set forth in the sequence listing of PCT/US06/038236.

It will be appreciated that a regulatory region may meet criteria for one classification based on its activity in one plant species, and yet meet criteria for a different classification based on its activity in another plant species.

Broadly Expressing Promoters

A promoter can be said to be “broadly expressing” when it promotes transcription in many, but not necessarily all, plant tissues. For example, a broadly expressing promoter can promote transcription of an operably linked sequence in one or more of the shoot, shoot tip (apex), and leaves, but weakly or not at all in tissues such as roots or stems. As another example, a broadly expressing promoter can promote transcription of an operably linked sequence in one or more of the stem, shoot, shoot tip (apex), and leaves, but can promote transcription weakly or not at all in tissues such as reproductive tissues of flowers and developing seeds. Non-limiting examples of broadly expressing promoters that can be included in the nucleic acid constructs provided herein include the p326, YP0144, YP0190, p13879, YP0050, p32449, 21876, YP0158, YP0214, YP0380, PT0848, and PT0633 promoters. Additional examples include the cauliflower mosaic virus (CaMV) 35S promoter, the mannopine synthase (MAS) promoter, the 1′ or 2′ promoters derived from T-DNA of Agrobacterium tumefaciens, the figwort mosaic virus 34S promoter, actin promoters such as the rice actin promoter, and ubiquitin promoters such as the maize ubiquitin-1 promoter. In some cases, the CaMV 35S promoter is excluded from the category of broadly expressing promoters.

Root Promoters

Root-active promoters confer transcription in root tissue, e.g., root endodermis, root epidermis, or root vascular tissues. In some embodiments, root-active promoters are root-preferential promoters, i.e., confer transcription only or predominantly in root tissue. Root-preferential promoters include the YP0128, YP0275, PT0625, PT0660, PT0683, and PT0758 promoters. Other root-preferential promoters include the PT0613, PT0672, PT0688, and PT0837 promoters, which drive transcription primarily in root tissue and to a lesser extent in ovules and/or seeds. Other examples of root-preferential promoters include the root-specific subdomains of the CaMV 35S promoter (Lam et al., Proc. Natl. Acad. Sci. USA, 86:7890-7894 (1989)), root cell specific promoters (reported by Conkling et al., Plant Physiol., 93:1203-1211 (1990)), and the tobacco RD2 promoter.

Maturing Endosperm Promoters

In some embodiments, promoters that drive transcription in maturing endosperm can be useful. Transcription from a maturing endosperm promoter typically begins after fertilization and occurs primarily in endosperm tissue during seed development and is typically highest during the cellularization phase. Most suitable are promoters that are active predominantly in maturing endosperm, although promoters that are also active in other tissues can sometimes be used. Non-limiting examples of maturing endosperm promoters that can be included in the nucleic acid constructs provided herein include the napin promoter, the Arcelin-5 promoter, the phaseolin promoter (Bustos et al., Plant Cell, 1(9):839-853 (1989)), the soybean trypsin inhibitor promoter (Riggs et al., Plant Cell, 1(6):609-621 (1989)), the ACP promoter (Baerson et al., Plant Mol. Biol., 22(2):255-267 (1993)), the stearoyl-ACP desaturase promoter (Slocombe et al., Plant Physiol., 104(4):167-176 (1994)), the soybean α′ subunit of β-conglycinin promoter (Chen et al., Proc. Natl. Acad. Sci. USA, 83:8560-8564 (1986)), the oleosin promoter (Hong et al., Plant Mol. Biol., 34(3):549-555 (1997)), and zein promoters, such as the 15 kD zein promoter, the 16 kD zein promoter, 19 kD zein promoter, 22 kD zein promoter and 27 kD zein promoter. Also suitable are the Osgt-1 promoter from the rice glutelin-1 gene (Zheng et al., Mol. Cell. Biol., 13:5829-5842 (1993)), the beta-amylase promoter, and the barley hordein promoter. Other maturing endosperm promoters include the YP0092, PT0676, and PT0708 promoters.

Photosynthetic Tissue Promoters

Promoters active in photosynthetic tissue confer transcription in green tissues such as leaves and stems. Most suitable are promoters that drive expression only or predominantly in such tissues. Examples of such promoters include the ribulose-1,5-bisphosphate carboxylase (RbcS) promoters such as the RbcS promoter from eastern larch (Larix laricina), the pine cab6 promoter (Yamamoto et al., Plant Cell Physiol., 35:773-778 (1994)), the Cab-1 promoter from wheat (Fejes et al., Plant Mol. Biol., 15:921-932 (1990)), the CAB-1 promoter from spinach (Lubberstedt et al., Plant Physiol., 104:997-1006 (1994)), the cab1R promoter from rice (Luan et al., Plant Cell, 4:971-981 (1992)), the pyruvate orthophosphate dikinase (PPDK) promoter from corn (Matsuoka et al., Proc. Natl. Acad. Sci. USA, 90:9586-9590 (1993)), the tobacco Lhcb1*2 promoter (Cerdan et al., Plant Mol. Biol., 33:245-255 (1997)), the Arabidopsis thaliana SUC2 sucrose-H+ symporter promoter (Truernit et al., Planta, 196:564-570 (1995)), and thylakoid membrane protein promoters from spinach (psaD, psaF, psaE, PC, FNR, atpC, atpD, cab, rbcS). Other photosynthetic tissue promoters include PT0535, PT0668, PT0886, YP0144, YP0380 and PT0585.

Vascular Tissue Promoters

Examples of promoters that have high or preferential activity in vascular bundles include YP0087, YP0093, YP0108, YP0022, and YP0080. Other vascular tissue-preferential promoters include the glycine-rich cell wall protein GRP 1.8 promoter (Keller and Baumgartner, Plant Cell, 3(10):1051-1061 (1991)), the Commelina yellow mottle virus (CoYMV) promoter (Medberry et al., Plant Cell, 4(2):185-192 (1992)), and the rice tungro bacilliform virus (RTBV) promoter (Dai et al., Proc. Natl. Acad. Sci. USA, 101(2):687-692 (2004)).

Inducible Promoters

Inducible promoters confer transcription in response to external stimuli such as chemical agents or environmental stimuli. For example, inducible promoters can confer transcription in response to hormones such as giberellic acid or ethylene, or in response to light or drought. Examples of drought-inducible promoters include YP0380, PT0848, YP0381, YP0337, PT0633, YP0374, PT0710, YP0356, YP0385, YP0396, YP0388, YP0384, PT0688, YP0286, YP0377, PD1367, and PD0901. Examples of nitrogen-inducible promoters include PT0863, PT0829, PT0665, and PT0886. Examples of shade-inducible promoters include PRO924 and PT0678. An example of a promoter induced by salt is rd29A (Kasuga et al., Nature Biotech 17: 287-291 (1999)).

Basal Promoters

A basal promoter is the minimal sequence necessary for assembly of a transcription complex required for transcription initiation. Basal promoters frequently include a “TATA box” element that may be located between about 15 and about 35 nucleotides upstream from the site of transcription initiation. Basal promoters also may include a “CCAAT box” element (typically the sequence CCAAT) and/or a GGGCG sequence, which can be located between about 40 and about 200 nucleotides, typically about 60 to about 120 nucleotides, upstream from the transcription start site.

Stem Promoters

A stem promoter may be specific to one or more stem tissues or specific to stem and other plant parts. Stem promoters may have high or preferential activity in, for example, epidermis and cortex, vascular cambium, procambium, or xylem. Examples of stem promoters include YP0018 which is disclosed in US20060015970 and CryIA(b) and CryIA(c) (Braga et al., Journal of new seeds 5:209-221 (2003)).

Other Promoters

Other classes of promoters include, but are not limited to, shoot-preferential, callus-preferential, trichome cell-preferential, guard cell-preferential such as PT0678, tuber-preferential, parenchyma cell-preferential, and senescence-preferential promoters. Promoters designated YP0086, YP0188, YP0263, PT0758, PT0743, PT0829, YP0119, and YP0096, as described in the above-referenced patent applications, may also be useful.

Other Regulatory Regions

A 5′ untranslated region (UTR) can be included in nucleic acid constructs described herein. A 5′ UTR is transcribed, but is not translated, and lies between the start site of the transcript and the translation initiation codon and may include the +1 nucleotide. A 3′ UTR can be positioned between the translation termination codon and the end of the transcript. UTRs can have particular functions such as increasing mRNA stability or attenuating translation. Examples of 3′ UTRs include, but are not limited to, polyadenylation signals and transcription termination sequences, e.g., a nopaline synthase termination sequence.

It will be understood that more than one regulatory region may be present in a recombinant polynucleotide, e.g., introns, enhancers, upstream activation regions, transcription terminators, and inducible elements. Thus, for example, more than one regulatory region can be operably linked to the sequence of a polynucleotide encoding a truncated lignin-modulating polypeptide.

Regulatory regions, such as promoters for endogenous genes, can be obtained by chemical synthesis or by subcloning from a genomic DNA that includes such a regulatory region. A nucleic acid comprising such a regulatory region can also include flanking sequences that contain restriction enzyme sites that facilitate subsequent manipulation.

### C. Co-Cultivation

Miscanthus transformation can include co-cultivation of induced calli and Agrobacterium containing an exogenous nucleic acid, under conditions wherein the Agrobacterium transfers the exogenous nucleic acid to a plant cell. Generally, co-cultivation can include inoculating a suspension of calli with an appropriate concentration of Agrobacterium under conditions wherein the calli become infected by Agrobacterium, and culturing infected calli to produce transformed calli. In some cases, induced calli can include embryogenic and non-embryogenic calli. In some cases, an inoculation step can include incubating induced calli with an appropriate concentration of Agrobacterium in co-cultivation medium for 30 minutes, and then transferring infected calli to medium-soaked filter paper in a petri dish for 5-7 days of co-cultivation.

A co-cultivation medium for transforming embryogenic Miscanthus calli can include a basal medium (e.g., MS or N6); B5-vitamins; sugars (e.g., maltose and/or sucrose); amino acids (e.g., L-cysteine); auxins and/or cytokinins (e.g., 2,4-D and/or kinetin); and, an activator of Agrobacterium virulence genes (e.g., a vir-inducing factor, such as acetosyringone). In some embodiments, a co-cultivation media can have a basic to acidic pH. In some embodiments, a co-cultivation medium can comprise about 1/10 to ½ MS basal medium, about 1/10 to ½ B5 vitamins, about 1 to 5% maltose, about 100 to 300 mg/L L-cysteine, about 1.0 to 5.0 mg/L 2,4-D, about 0.25 to 1.00 mg/L kinetin, and about 100 to 400 μM acetosyringone (e.g., 125 to 400 μM, 150 to 400 μM, 200 to 400 μM, 250 to 400 μM, 275 to 350 μM, 300 to 400 μM, or 350 to 400 μM acetosyringone), at about pH 5.20 to 5.50. In some embodiments, a co-cultivation medium can comprise about 2,4-D and kinetin in a ratio of about 4:1 or 5:1. In some embodiments, co-cultivation media can comprise 1/10 MS basal medium, 1/10 B5 vitamins, 3% maltose, 200 mg/L L-cysteine, 2 mg/L 2,4-D, 0.5 mg/L kinetin, and 300 μM acetosyringone, at pH 5.4. In some embodiments, a co-cultivation media comprises at least 200, 250, 300, 350, or 400 μM acetosyringone. In some embodiments, a co-cultivation media comprises at least about 300 μM acetosyringone. In some embodiments, a co-cultivation media comprises about 210, 225, 250, 275, 300, 325, 350, 375, 400, 425, 450, 475, 500, 525, 550, 575, or 600 μM acetosyringone. In some embodiments, a co-cultivation medium can include glucose (e.g., 1% glucose) and/or L-glutamine (e.g., 50 mg/L glutamine).

Co-cultivation conditions, such as time, temperature, PAR, and photoperiod, can be modified as desired in order to promote transformation. In some embodiments, an appropriate duration for incubation with infected calli is from 10 to 60 minutes, e.g., 15 to 45 minutes, 20 to 40 minutes, or 30 minutes. In some embodiments, an appropriate duration for co-cultivation can be from about 4, 5, 6, 7, 8, or 9 days. In some embodiments, an appropriate temperature for co-cultivation can be about 20, 21, 22, 23, 24, 25, 26, or 27° C. In some embodiments, an appropriate PAR for co-cultivation can be a PPFD of about 0 to 5 μmol/m²/s. In some embodiments, an appropriate photoperiod can be about 10, 14, 16, 18, 20, 22, or 24 hours of light per day.

Calli can be transferred to a recovery medium following co-cultivation. A recovery medium can include an agent for inhibiting the growth of Agrobacterium, such as, without limitation, TIMENTIN® (ticarcillin disodium and clavulanate potassium), cefotaxime, carbenincillin, and clavamox (amoxicillin and lithium clavulanate). In some embodiments, a recovery medium can comprise a basal media, a micronutrient, a nutrient, an amino acid, a sugar, a gelling agent, an auxin and/or a cytokinin. In some embodiments, a recovery medium can comprise a basal medium, such as N6, NB, or Gamborg B-5, about 0.75 to 1.00 mg/L KI, about 0.025 to 0.10 mg/L CoCI₂.6H₂O, about 0.025 to 0.20 mg/L CuSO₄.5H₂O, about 0.10 to 0.30 mg/L NaMoO₄.2H₂O, about 1.0 to 3.0 mg/L glycine, about 50.0 to 200.0 mg/L myo-inositol, 0.10 to 10.0 mg/L thiamine HCl, 0.50 to 1.00 mg/L pyridoxine HCl, 0.50 to 1.0 mg/L nicotinic acid, 0.5 to 2.0 g/L casamino acid, 3.0 to 5.0 mg/L 2,4-D, 0.50 to 1.0 mg/L BA, 1 to 5% maltose, 6.0 to 8.0 g/L agar (Phytotechnology Laboratories™), and 100 to 250 mg/L TIMENTIN®, at pH 5.6 to 5.8. In some embodiments, a recovery medium can comprise 2,4-D and BA in a ratio of about 4:1 or 5:1. In some embodiments, a recovery medium can comprise N6, about 0.8 mg/L KI, about 0.025 mg/L CoCI₂.6H₂O, about 0.025 mg/L CuSO₄.5H₂O, about 0.25 mg/L NaMoO₄.2H₂O, about 2 mg/L glycine, about 100 mg/L myo-inositol, about 5 mg/L thiamine HCl, about 1 mg/L pyridoxine HCl, about 1 mg/L nicotinic acid, about 1 g/L casamino acid, about 4 mg/L 2,4-D, about 1.0 mg/L BA, about 3% maltose, about 7.0 g/L agar or agarose, and about 125 mg/L TIMENTIN® or Carbenicillin, at about pH 5.8. In some embodiments, amino acids such as proline and/or asparagine are included in a recovery medium. For example, at least 1 g/L such as 1 g/L, 1.25 g/L, 1.5 g/L, 1.75 g/L, or 2 g/L of proline and/or asparagine can be included in a recovery medium.

### D. Selection of Transformed Embryogenic Calli

A transformed cell, or callus, can be identified and isolated by selecting or screening calli for a phenotypes conferred by a selectable or screenable marker. For example, Miscanthus cells transformed according to the methods described herein can be identified using suitable molecular biological techniques, such as Southern analysis or PCR amplification, for detection of a polynucleotide comprising a nucleic acid sequence of an exogenous nucleic acid; northern blots, 51 RNase protection, primer-extension, or RT-PCR amplification for detecting RNA transcripts from the expression of an exogenous nucleic acid; enzymatic assays for detecting enzyme or ribozyme activity of polypeptides and polynucleotides encoded by an exogenous nucleic acid; and, protein gel electrophoresis, western blots, immunoprecipitation, and enzyme-linked immunoassays to detect polypeptides encoded by an exogenous nucleic acid. Other techniques such as in situ hybridization, enzyme staining, and immunostaining also can be used to detect the presence or expression of polypeptides and/or polynucleotides encoded by an exogenous nucleic acid.

Embryogenic calli can be screened for transformed calli using a selection medium comprising a recovery media supplemented with a selective agent, to which transformed calli are resistant due to expression of a selectable marker. For example, a suitable selection agent can be kanamycin, neomycin, paromomycin, butirosin, gentamycin B or geneticin, if the exogenous nucleic acid comprises a selectable marker encoding an nptII polypeptide. For example, a selection medium can comprise about 75 to 150 mg/L paromomycin, e.g., 100 mg/L paromomycin.

The conditions for selection of transformed calli can be modified as desired in order to identify and isolate stably transformed embryogenic calli capable of regeneration. Thus, in some embodiments, calli are cultured on a selection medium for about 5, 6, 7, 8, or 9 weeks. In some embodiments, the selection is carried out at about 22, 23, 24, 25, 26, or 27° C. In some embodiments, selection includes PAR with a PPFD of about 0-5 μmol/m²/s. In some embodiments, selection includes a photoperiod of about 10, 14, 16, 18, 20, 22, or 24 hours of light per day.

The transformation efficiency of the methods described herein ranges from 1% up to 70%, determined by dividing the number of stably transformed calli by the total number of calli subjected to the co-cultivation. For example, the transformation efficiency can be 1, 2, 3, 4, 5, 6, 7, 8, 9, 10, 12, 14, 16, 18, 20, 22, 24, 25, 30, 35, 40, 45, 50, 55, 60, 65, or 70% using the methods and compositions described herein. Typically, transformation efficiency is between about 1 and 15%, e.g., about 5 to about 15%. Transformation efficiencies of about 20 to about 30% can be obtained by including components such as acetosyringone (e.g., at least 200 μM acetosyringone) in the co-cultivation medium and amino acids such as proline and/or asparagine (e.g., at least 1 g/L) in the recovery and callus selection media.

### E. Miscanthus Regeneration

The methods provided herein can include regeneration of Miscanthus plants from transformed embryogenic calli. For example, regeneration can include growing shoots in/on a regeneration media, and rooting shoots in/on a rooting media to form plantlets. In some cases, a selection agent can incorporated into the media to confirm that regenerated plants are transformed.

Shoot Formation

Transformed embryogenic calli can be transferred to a regeneration media to allow for shoot formation. In some embodiments, a regeneration medium can comprise a basal medium, a salt, a vitamin, a nutrient, a micronutrient, a auxin and/or a cytokinin, a sugar, an amino acid, an antibiotic, and a gelling agent. In some embodiments, a regeneration medium can comprise MS or N6 basal salts, B5 vitamins, about 0.5 to 3.0 mg/L BA, about 0.05 to 1.0 mg/L naphthalene acetic acid (NAA), about 1 to 2% maltose, about 25 to 100 mg/L L-glutamine, about 1 to 3% sucrose, about 6.0 to 8.0 g/L agar, about 50 to 100 mg/L paromomycin, and about 125 to 250 mg/L TIMENTIN®, at about pH 5.6 to 5.8. In some embodiments, a regeneration medium can comprise BA and NAA in a ratio of about 10:1 or 6:1. In some embodiments, a regeneration medium can comprise MS basal salts, B5 vitamins, about 2 mg/L BA, about 0.1 mg/L NAA, about 1% maltose, about 50 mg/L L-glutamine, about 2% sucrose, about 7 g/L agar, about 50 mg/L paromomycin, and about 125 mg/L TIMENTIN®, at about pH 5.8.

Regeneration conditions can be adapted to optimize for shoot formation. For example, conditions, such as time, temperature, PAR, and photoperiod, can be varied to optimize production of transformed plants. In some embodiments, shoot formation can take about 2, 3, 4, 5, or 6 weeks. In some embodiments, regeneration can be carried out at about 22, 23, 24, 25, 26, 27, or 28° C. In some embodiments, regeneration can include PAR with a PPFD of about 35-75 μmol/m²/s. In some embodiments, a photoperiod during regeneration can be about 10, 14, 16, 18, 20, 22, or 24 hours of light per day.

Root Formation

After transformed embryogenic calli have formed shoots, the calli can be transferred to a rooting media. In some embodiments, a rooting media can comprise a basal medium, a salt, a nutrient, an amino acid, a sugar, an auxin and/or cytokinin, and a gelling agent. In some embodiments, a rooting media can comprise about ½ to 1×MS basal salts, about 1.00 to 3.00 mg/L glycine, about 0.10 to 10.0 mg/L thiamine, about 0.50 to 1.0 mg/L pyridoxine, about 0.50 to 1.0 mg/L nicotinic acid, about 50.0 to 200.0 mg/L myo-inositol, about 1 to 2% sucrose, about 0.5 to 1.0 mg/L NAA, about 125 to 250 mg/L TIMENTIN®, and about 2 to 3 g/L GELRITE®, at about pH 5.6 to 5.9. In some embodiments, a rooting media can comprise about ½ MS basal salts, about 0.2 mg/L glycine, about 0.5 mg/L thiamine, about 1.0 mg/L pyridoxine, about 1.0 mg/L nicotinic acid, about 100 mg/L myo-inositol, about 1.5% sucrose, about 0.5 mg/L naphthalene acetic acid NAA, about 125 mg/L TIMENTIN®, and about 2.5 g/L GELRITE®, at about pH 5.8.

The conditions for regeneration can be adapted to promote root formation. For example, time, temperature, PAR, and photoperiod can be varied to optimize formation of roots. In some embodiments, root formation can take about 2, 3, 4, 5, or 6 weeks. In some embodiments, root formation can be carried out at about 22, 23, 24, 25, 26, 27, or 28° C. In some embodiments, root formation can include PAR with a PPFD of about 35 to 75 μmol/m²/s. In some embodiments, root formation includes a photoperiod of about 10, 14, 16, 18, 20, 22, or 24 hours of light per day.

### II. MISCANTHUS PLANTS

Transgenic Miscanthus plants and cells are described herein. For example, a transgenic Miscanthus plant can comprise at least one exogenous nucleic acid described herein. For example, a transgenic plant or cell can comprise at least one T-DNA border sequence, or a functional analogue of a T-DNA border from a plant (e.g. a P-DNA border as described in Rommens et al., “Plant-Derived Transfer DNAs” Plant Physiology, 139: 1338-1349 (2005)). In some cases, a plant or plant cell can be transiently transformed or stably transformed.

When transiently transformed plant cells are used, a nucleotide sequence encoding a screenable marker can be included in the transformation procedure. An assay for screenable marker activity or expression can be performed at a suitable time after transformation. A suitable time for conducting a screening assay can be about 1-21 days after transformation, e.g., about 1-14 days, about 1-7 days, or about 1-3 days. Transient expression assays can be used to confirm expression of a transgene, which has not been previously confirmed in particular recipient cells.

Transgenic plant cells used in methods described herein can constitute part or all of a whole Miscanthus plant. Such plants can be grown in a manner suitable for the species under consideration, either in a growth chamber, a greenhouse, or in a field. Transgenic plants can be bred as desired for a particular purpose, e.g., to introduce a recombinant nucleic acid into other lines, to transfer a recombinant nucleic acid to other species, or for further selection of other desirable traits. Alternatively, transgenic plants can be propagated vegetatively. As used herein, a transgenic plant also refers to progeny of an initial transgenic plant provided the progeny inherits the transgene. Seeds produced by a transgenic plant can be grown and then selfed (or outcrossed and selfed) to obtain seeds homozygous for the nucleic acid construct.

A transformed Miscanthus tissue or plant can be identified and isolated by screening or selecting for particular traits or activities, e.g., those encoded by a screenable or selectable marker. In some cases, the transformed Miscanthus plants can be characterized by the presence of a transferred exogenous nucleic acid comprising a nucleotide sequence encoding a polypeptide, or transcription product of interest, flanked by at least one T-DNA border, inserted within the genome of the Miscanthus plant. For example, a transformed Miscanthus plant comprising an exogenous nucleic acid from an NB4KanEGFP plasmid can express an enhanced GFP polypeptide and have resistance to antibiotics such as kanamycin, neomycin and paromomycin.

Identification of transgenic Miscanthus plants, produced according to the methods described herein, can include physical and biochemical screening techniques. For example, Southern analysis or PCR amplification for detection of a polynucleotide; northern blots, S1 RNase protection, primer-extension, or RT-PCR amplification for detecting RNA transcripts; enzymatic assays for detecting enzyme or ribozyme activity of polypeptides and polynucleotides; and protein gel electrophoresis, western blots, immunoprecipitation, and enzyme-linked immunoassays to detect polypeptides. Other techniques such as in situ hybridization, enzyme staining, and immunostaining also can be used to detect the presence or expression of polypeptides and/or polynucleotides, can be used to identify a transgenic Miscanthus plant comprising an exogenous nucleic acid. After a the presence of a stably incorporated exogenous nucleic acid is confirmed in a regenerated plant, the exogenous nucleic acid can be introduced into other plants using, for example, standard breeding techniques.

A Miscanthus plant transformed by the methods described herein can be of a number of species, variety, or clone of the genus Miscanthus or a hybrid thereof. A Miscanthus plant used in the methods described herein can be derived from germplasm that is, for example, wild type, ornamental, fiber, feed, forage, or fuel cultivars, including, but not limited to, Miscanthus x giganteus, Miscanthus sinensis, Miscanthus x ogiformis, Miscanthus floridulus, Miscanthus transmorrisonensis, Miscanthus oligostachyus, Miscanthus nepalensis, Miscanthus sacchariflorus, Miscanthus x giganteus ‘Amuri’, Miscanthus x giganteus ‘Nagara’, Miscanthus x giganteus ‘Illinois’, Miscanthus sinensis var. ‘Goliath’, Miscanthus sinensis var. ‘Roland’, Miscanthus sinensis var. ‘Africa’, Miscanthus sinensis var. ‘Fern Osten’, Miscanthus sinensis var. gracillimus, Miscanthus sinensis var. variegates, Miscanthus sinensis var. purpurascens, Miscanthus sinensis var. ‘Malepartus’, Miscanthus sacchariflorus var. ‘Robusta’, Miscanthus sinensis var. ‘Silberfedher’ (aka. Silver Feather), Miscanthus transmorrisonensis, Miscanthus condensatus, Miscanthus yakushimanum, Miscanthus var. ‘Alexander’, Miscanthus var. ‘Adagio’, Miscanthus var. ‘Autumn Light’, Miscanthus var. ‘Cabaret’, Miscanthus var. ‘Condensatus’, Miscanthus var. ‘Cosmopolitan’, Miscanthus var. ‘Dixieland’, Miscanthus var. ‘Gilded Tower’ (U.S. Pat. No. PP14,743), Miscanthus var. ‘Gold Bar’ (U.S. Pat. No. PP15,193), Miscanthus var. ‘Gracillimus’, Miscanthus var. ‘Graziella’, Miscanthus var. ‘Grosse Fontaine’, Miscanthus var. ‘Hinjo aka Little Nicky’™, Miscanthus var. ‘Juli’, Miscanthus var. ‘Kaskade’, Miscanthus var. ‘Kirk Alexander’, Miscanthus var. ‘Kleine Fontaine’, Miscanthus var. ‘Kleine Silberspinne’ (aka. ‘Little Silver Spider’), Miscanthus var. ‘Little Kitten’, Miscanthus var. ‘Little Zebra’ (U.S. Pat. No. PP13,008), Miscanthus var. ‘Lottum’, Miscanthus var. ‘Malepartus’, Miscanthus var. ‘Morning Light’, Miscanthus var. ‘Mysterious Maiden’ (U.S. Pat. No. PP16,176), Miscanthus var. ‘Nippon’, Miscanthus var. ‘November Sunset’, Miscanthus var. ‘Parachute’, Miscanthus var. ‘Positano’, Miscanthus var. ‘Puenktchen’(aka ‘Little Dot’), Miscanthus var. ‘Rigoletto’, Miscanthus var. ‘Sarabande’, Miscanthus var. ‘Silberpfeil’ (aka. Silver Arrow), Miscanthus var. ‘Silverstripe’, Miscanthus var. ‘Super Stripe’ (U.S. Pat. No. PP18,161), Miscanthus var. ‘Strictus’, or Miscanthus var. ‘Zebrinus’.

In certain embodiments, a plant propagated by the methods described herein can be a hybrid of different species, varieties of a specific species, or clones of a variety (e.g., Saccharum sp. x Miscanthus sp.).

### III. PLANT BREEDING

Those regenerated transformed Miscanthus plants from fertile species can be entered into a plant breeding program, such as a marker-associated breeding program. For example, an exogenous nucleic acid (transgene) can be introgressed into desired germplasm, or a transgenic plant can be used in selection, breeding, and/or hybridization.

Plant breeding methods include use of molecular markers. Genetic polymorphisms that are useful in plant breeding and include simple sequence repeats (SSRs, or microsatellites), rapid amplification of polymorphic DNA (RAPDs), single nucleotide polymorphisms (SNPs), amplified fragment length polymorphisms (AFLPs) and restriction fragment length polymorphisms (RFLPs). SSR polymorphisms can be identified, for example, by making sequence specific probes and amplifying template DNA from individuals in the population of interest by PCR. If the probes flank an SSR in the population, PCR products of different sizes will be produced (see, e.g., U.S. Pat. No. 5,766,847). Alternatively, SSR polymorphisms can be identified by using PCR product(s) as a probe against Southern blots from different individuals in the population (see, U. H. Refseth et al., Electrophoresis 18: 1519 (1997)). The identification of RFLPs is discussed, for example, in Alonso-Blanco et al. (Methods in Molecular Biology, vol. 82, “Arabidopsis Protocols”, pp. 137-146, J. M. Martinez-Zapater and J. Salinas, eds., c. 1998 by Humana Press, Totowa, N.J.); Burr (“Mapping Genes with Recombinant Inbreds”, pp. 249-254, in Freeling, M. and V. Walbot (Ed.), The Maize Handbook, c. 1994 by Springer-Verlag New York, Inc.: New York, N.Y., USA; Berlin Germany; Burr et al. Genetics 118: 519 (1998); and Gardiner, J. et al., Genetics 134: 917 (1993)). The identification of AFLPs is discussed, for example, in EP 0 534 858 and U.S. Pat. No. 5,878,215.

Techniques suitable for use in a plant breeding program include, without limitation, backcrossing, mass selection, pedigree breeding, bulk selection, crossing to another population and recurrent selection. These techniques can be used alone or in combination with one or more other techniques in a breeding program. For example, each identified plant can be selfed or crossed a different plant to produce seed which can be germinated to form progeny plants. At least one such progeny plant can be selfed or crossed with a different plant to form a subsequent progeny generation. The breeding program can repeat the steps of selfing or outcrossing for an additional 0 to 5 generations as appropriate in order to achieve the desired uniformity and stability in the resulting plant line, which retains the transgene. In most breeding programs, analysis for the particular polymorphic allele will be carried out in each generation, although analysis can be carried out in alternate generations if desired.

### IV. EXAMPLES

### Example 1

### Miscanthus Transformation

M. sinensis germinated seeds and immature inflorescence (IF) from M. x giganteus and the dwarf variety M. sacchariflorus ‘Robusta’ were surface-sterilized with 20% (v/v) Clorox (commercial bleach) and 0.1% liquinox (surfactant) for 20 minutes. The tissue was rinsed 5 times with sterile MilliQH₂O. IF were cut into 1.0 cm segments. Young shoots with 1-2 leaves were collected from in vitro plants, which were maintained on Miscanthus Rooting medium (MRt) (Table 1) in a sterile box. The outermost leaf and leaf sheath were removed.

TABLE 1
Media formulations for Miscanthus transformation

| Media Components | MC | | MLC | MRR | | MCS | | MR tp | MRt | |
|---|---|---|---|---|---|---|---|---|---|---|
| Basal medium | N6 | | 1/10 MS | N6 | | N6 | | MS | 1/2 MS | |
| KI | 0.8 | mg/L | | 0.8 | mg/L | 0.8 | mg/L | | | |
| CoCl₂•6 H₂O | 0.025 | mg/L | | 0.025 | mg/L | 0.025 | mg/L | | | |
| CuSO₄•5 H₂O | 0.025 | mg/L | | 0.025 | mg/L | 0.025 | mg/L | | | |
| NaMoO₄•2 H₂O | 0.25 | mg/L | | 0.25 | mg/L | 0.25 | mg/L | | | |
| Amino acid | 2 mg/L | | 200 mg/L | 2 mg/L | | 2 mg/L | | 50 mg/L | 0.2 mg/L | |
| | glycine | | cysteine | glycine | | glycine | | L-glutamine | glycine | |
| Casamino Acid | 1 | g/L | | 1 | g/L | 1 | g/L | | 1 | g/L |
| Myo-inositol | 100 | mg/L | | 100 | mg/L | 100 | mg/L | | 100 | mg/L |
| B5 Vitamins | | | 1/10 | | | | | 1X | | |
| Thiamine | 5 | mg/L | | 5 | mg/L | 5 | mg/L | | 0.5 | mg/L |
| Pyridoxine | 1 | mg/L | | 1 | mg/L | 1 | mg/L | | 0.1 | mg/L |
| Nicotinic Acid | 1 | mg/L | | 1 | mg/L | 1 | mg/L | | 1 | mg/L |

| Auxin | 2 mg/L 2,4-D | 2 mg/L 2,4-D | 4 mg/L 2,4-D | 4 mg/L 2,4-D | 0.1 mg/L NAA | 0.5 mg/L NAA |
|---|---|---|---|---|---|---|
| Cytokinin | 0.5 mg/L | 0.5 mg/L | 1 mg/L BA | 1 mg/L BA | 2 mg/L BA | 0.5 mg/L |
| | Kinetin | Kinetin | | | | Kinetin |
| Sugar | 3% Maltose | 3% Maltose | 3% Maltose | 3% Maltose | 1% Maltose | 1.5% Sucrose |
| | | | | | 2% Sucrose | |
| Gelling agent | 3.5 g/L | | 7 g/L Agar | 7 g/L Agar | 7 g/L Agar | 2.5 g/L |
| | GELRITE ® | | (Phytotechnology | | | GELRITE ® |
| | | | Laboratories ™) | | | |
| pH | 5.8 | 5.4 | 5.8 | 5.8 | 5.8 | 5.8 |
| Supplement | | 300 μM | 125 mg/L | 100 mg/L | 50 mg/L | 125 mg/L |
| | | Aceto- | TIMENTIN ® | Paromomycin | Paromomycin | TIMENTIN ® |
| | | syringone | | 125 mg/L | 125 mg/L | |
| | | | | TIMENTIN ® | TIMENTIN ® | |

Prepared IF, young shoots, and mature seeds were transferred to Miscanthus Callus Induction medium (MC) (Table 1). Primary calli were observed in 1-2 weeks. Calli were subcultured on the same medium every two weeks until translucent spherical embryogenic calli (EC) were formed among friable non-embryogenic callus. EC were subcultured every two weeks. Only EC were used for Agrobacterium infection. To determine plant regeneration capacity, calli were transferred to the Regeneration Medium (MR) for 2-4 weeks. MR consisted of MS basal salts, B5 vitamins, 2 mg/L BA, 0.1 mg/L NAA, 1% maltose, 50 mg/L L-glutamine, 2% sucrose, and 7 g/L agar (Phytotechnology Laboratories™), at pH 5.8. Subculture was performed when necessary. To keep regenerable and transformable calli constantly available for transformation, callus was initiated from IF every 3-6 months.

A culture of Agrobacterium cells comprising an NB4KAN:p326-EGFP construct (FIG. 4), which had a nucleotide sequence encoding a selectable marker (nptII), a promoter, and a GFP polypeptide, was prepared by inoculating Agrobacterium (strain EHA105) from glycerol stock into 2 mL YEB liquid medium plus antibiotics (5 g/L Beef extract, 5 g/L Peptone, 1 g/L Yeast extract, 5 g/L sucrose, 2 mL/L 1M MgSO₄), and incubated overnight on a 28° C. shaker. The culture was re-inoculated into 5 mL YEB plus antibiotics and cultured on a 28° C. shaker overnight. The culture was spun down in a 2.0 mL Eppendorf® tube at 10,000 rpm for 3 minutes using a micro-centrifuge. The pellet was re-suspended in Miscanthus Liquid Co-cultivation medium (MLC) (Table 1). The Agrobacterium suspension was incubated on 22° C. shaker at 110 rpm in the dark until ready to use for infection. EC were added to the bacterial suspension, and incubated on a 22° C. shaker at 110 rpm in the dark for 30 minutes. The EC were transferred to a sterile wipe (Kimwipes®), and blotted dry in a petri dish. EC were spread on three sterile filter papers (Whatman®) wetted with 2.1 mL MLC in 100×20 mm petri dishes. The dishes were sealed with plastic wrap.

Dishes were incubated (co-cultivated) at 25° C. in the dark in a growth chamber for 5-7 days. Calli were recovered after 5-7 days of co-cultivation. Calli were rinsed with 20-30 mL of sterile MilliQH₂O at least one time, and rinsed once with 15-20 mL of 250 mg/L sterile TIMENTIN® solution. Calli were blotted briefly with a sterile wipe in a petri dish. Small calli clusters were transferred onto Miscanthus Recovery medium (MRR) (Table 1) and cultured at 25° C. in the dark of a growth chamber for 5-7 days.

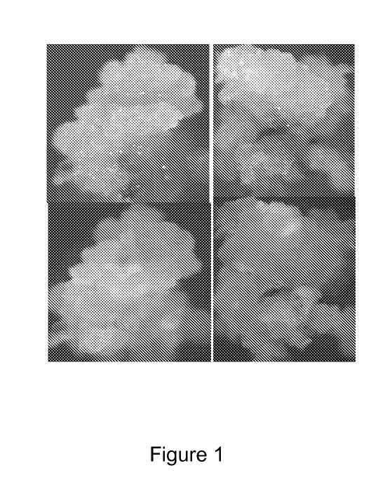

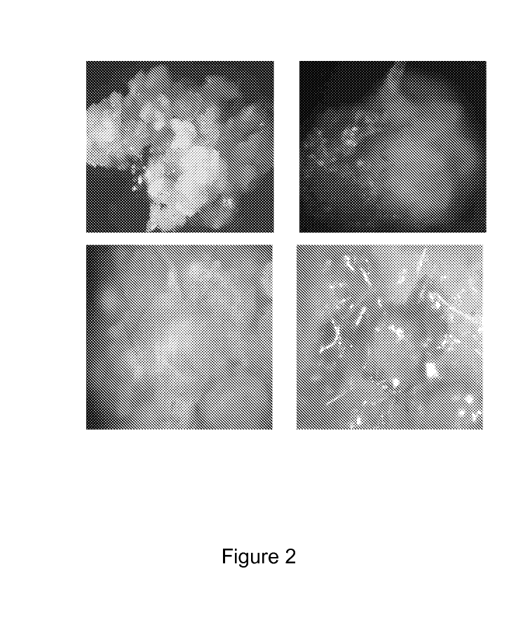

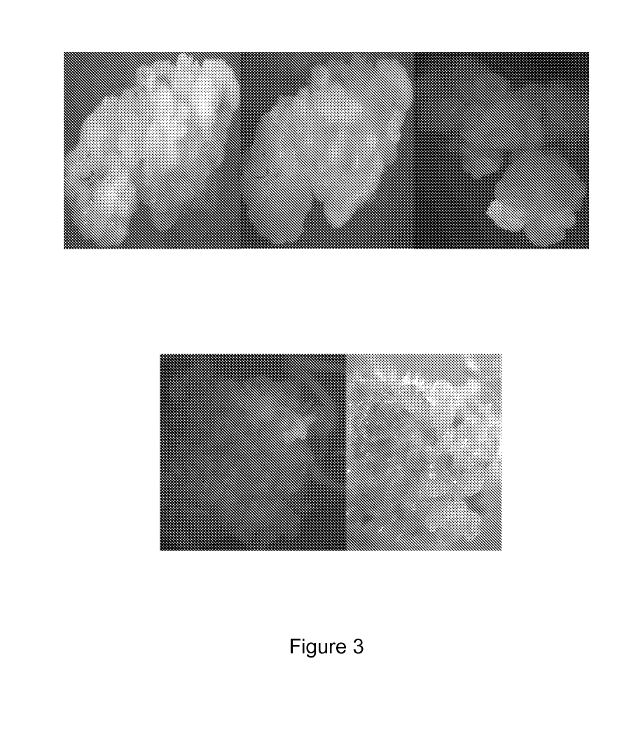

After the recovery period, all calli were transferred to Miscanthus Callus Selection medium (MCS) and incubated at 25° C. in the dark of a growth chamber. MCS consisted of MRR, supplemented with 100 mg/L paromomycin. Calli were subcultured every 14 days under the same conditions, until transgenic calli were confirmed by screening for GFP expression. FIGS. 1-3 show GFP expressing calli 60 days after infection of M. sinensis, M. x giganteus, and M. sacchariflorus ‘Robusta’, respectively. The transformation efficiency was about 5 to 15%.

Paromomycin resistant calli were transferred onto Miscanthus Regeneration medium (MR tp) (Table 1) and incubated at 27° C., under light, in a growth chamber with a 16 hour photoperiod. Regenerated plantlets were transferred onto Miscanthus Rooting medium (MRt) in a sterile box, and incubated at 27° C. under light, in a growth chamber with 16 hour photoperiod.

Well-rooted plants were transplanted into Soil mix 1 (Soil 1) and moved to a recovery room. Soil 1 consisted of 20% of Metro-Mix® 200, 70% of Sunshine® mix #5, and 10% vermiculite. Osmocote® (1 tablespoon for 4 liters of soil mix) and granular Marathon® (½ tablespoon for 4 liters of soil mix) were added. All plants were misted with water and covered with clear lids. The plastic covers were removed when plants had recovered for 1-2 days. Plants were moved to a greenhouse after 1 week. Plants were self-pollinated and T₁ progeny seeds were collected.

### Example 2

### Miscanthus Transformation

M. sinensis germinated seeds and immature inflorescence (IF) from M. x giganteus and the dwarf variety M. sacchariflorus ‘Robusta’ were transformed as described in Example 1, except that the media described in Table 2 were used in place of the media in Table 1 and Miscanthus EC were contacted with Agrobacterium as follows.

MLC liquid medium was added to a 50 ml conical tube in an amount sufficient to just cover the EC. The tube was heated at 43° C. for 3 minutes and allowed to cool to room temperature. The liquid was removed from the tube and the Agrobacterium suspension was added. The tube was incubated on a 22° C. shaker at 110 rpm in the dark for 30 minutes. The EC were transferred to a sterile wipe (Kimwipes®), and blotted dry in a petri dish. EC were spread on three sterile filter papers (Whatman®) wetted with 2.2 mL MLC in 100×20 mm petri dishes. The dishes were sealed with plastic wrap.

After the recovery period, all calli were transferred to MCS medium and incubated at 28° C. in the dark of a growth chamber. The transformation efficiency was about 20 to 30%.

TABLE 2
Media formulations for Miscanthus transformation

| Media | | | | | | | | |
|---|---|---|---|---|---|---|---|---|
| Components | MC | | MLC | | MRR | | MCS | |
| Basal medium | N6 | | 1/10 MS | | N6 | | N6 | |
| KI | 0.8 | mg/L | | | 0.8 | mg/L | 0.8 | mg/L |
| CoCl₂•6 H₂O | 0.025 | mg/L | | | 0.025 | mg/L | 0.025 | mg/L |
| CuSO₄•5 H₂O | 0.025 | mg/L | | | 0.025 | mg/L | 0.025 | mg/L |
| NaMoO₄•2 H₂O | 0.25 | mg/L | | | 0.25 | mg/L | 0.25 | mg/L |
| Amino acid | 2 | mg/L | 200 | mg/L | 2 | mg/L | 2 | mg/L |
| | glycine | | cysteine; | | glycine; | | glycine; | |
| | | | 50 | mg/L | 1 | gm/L | 1 | gm/L |
| | | | glutamine | | Asparagine; | | Asparagine; | |
| | | | | | 2 | gm/L | 2 | gm/L |
| | | | | | Proline | | Proline | |
| Casamino | 1 | g/L | | | 1 | g/L | 1 | g/L |
| Acid | | | | | | | | |
| Myo-inositol | 100 | mg/L | | | 100 | mg/L | 100 | mg/L |
| B5 Vitamins | | | 1/10 | | | | | |
| Thiamine | 5 | mg/L | | | 5 | mg/L | 5 | mg/L |
| Pyridoxine | 1 | mg/L | | | 1 | mg/L | 1 | mg/L |
| Nicotinic Acid | 1 | mg/L | | | 1 | mg/L | 1 | mg/L |
| Auxin | 2 | mg/L | 2 | mg/L | 4 | mg/L | 4 | mg/L |
| | 2,4-D | | 2,4-D | | 2,4-D | | 2,4-D | |
| Cytokinin | 0.5 | mg/L | 0.5 | mg/L | 1 | mg/L | 1 | mg/L |

| | BA | | BA | BA | | BA | |
|---|---|---|---|---|---|---|---|
| Sugar | 3% | | 3% | 3% | | 3% | |
| | Maltose | | Maltose; | Maltose | | Maltose | |
| | | | 1% | | | | |
| | | | Glucose | | | | |
| Gelling agent | 3.5 | g/L | | 7 | g/L | 7 | g/L |

| | GELRITE ® | | | Agarose | | Agarose | |
|---|---|---|---|---|---|---|---|
| pH | 5.8 | 5.4 | | 5.8 | | 5.8 | |
| Supplement | | 300 | μM | 125 | mg/L | 100 | mg/L |

| Aceto- | Carbenicillin | Paromomycin | |
|---|---|---|---|
| syringone | | 125 | mg/L |

Carbenicillin

The media used for regeneration, MR tp and MRt, were the same as those used in Example 1. Plants selected as described in Example 1 were transplanted to soil and moved to a greenhouse as described in Example 1.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of transforming Miscanthus, said method comprising
(a) contacting an embryogenic Miscanthus callus with Agrobacterium comprising an exogenous nucleic acid in a medium comprising 1/10 MS basal medium, 1/10 B5 vitamins, 3% maltose, 200 mg/L L-cysteine, 2 mg/L 2,4-D, 0.5 mg/L kinetin, and at least 300 μM acetosyringone, at pH 5.4 under conditions wherein said callus is infected by said Agrobacterium, wherein said exogenous nucleic acid comprises a nucleic acid sequence encoding a selectable marker conferring resistance to a selection agent; and
(b) selecting for at least one transformed callus, derived from step (a), in a medium containing said selection agent, wherein said selection medium inhibits the growth of said Agrobacterium.

2. The method of claim 1, wherein step (a) of said method further comprises culturing said Agrobacterium-infected callus on said medium for 5-7 days.

3. The method of claim 1, wherein said method further comprises culturing said callus derived from step (a) on a recovery medium comprising N6 basal medium, 0.8 mg/L KI, 0.025 mg/L CoCI₂.6H₂O, 0.025 mg/L CuSO₄.5H₂O, 0.25 mg/L NaMoO₄.2H₂O, 2 mg/L glycine, 100 mg/L myo-inositol, 5 mg/L thiamine HCl, 1 mg/L pyridoxine HCl, 1 mg/L nicotinic acid, 1 g/L casamino acid, 2 mg/L 2,4-D, 1.0 mg/L BA, 3% maltose, 125 mg/L TIMENTIN®, and 7.0 g/L agar at pH 5.8 for 5-7 days.

4. The method of claim 3, wherein said recovery medium further comprises 1 g/L asparagine and 2 g/L proline.

5. The method of claim 1, wherein said method further comprises inducing said embryogenic callus from Miscanthus tissue on an induction medium prior to said contacting step.

6. The method of claim 5, wherein said Miscanthus tissue is an immature inflorescence, a germinated seed, or a shoot apex.

7. The method of claim 6, wherein said Miscanthus tissue is an explant from a Miscanthus plant selected from the group consisting of Miscanthus sinensis, Miscanthus x giganteus, Miscanthus floridulus and Miscanthus sacchariflorus.

8. The method of claim 5, wherein said inducing step comprises subculturing primary calli on said induction medium every two weeks until said embryogenic callus is formed.

9. The method of claim 5, wherein said induction medium comprises 2,4-D and kinetin in a four to one ratio.

10. The method of claim 5, wherein said induction medium comprises N6 basal medium, 0.8 mg/L KI, 0.025 mg/L CoCI₂.6H₂O, 0.025 mg/L CuSO₄.5H₂O, 0.25 mg/L NaMoO₄.2H₂O, 2 mg/L glycine, 100 mg/L myo-inositol, 5 mg/L thiamine HCl, 1 mg/L pyridoxine HCl, 1 mg/L nicotinic acid, 1 g/L casamino acid, 2 mg/L 2,4-D, 0.5 mg/L kinetin, 3% maltose, and 3.5 g/L GELRITE®, at pH 5.8.

11. The method of claim 1, wherein said selectable marker confers herbicide resistance.

12. The method of claim 11, wherein said selectable marker is a phosphinothricin acetyltransferase polypeptide, a glyphosate resistant 3-enolpyruvylshikimate-5-phosphate synthate polypeptide or a glyphosate oxidoreducatase polypeptide.

13. The method of claim 1, wherein said selectable marker confers antibiotic resistance.

14. The method of claim 13, wherein said selectable marker is a neomycin phosphotransferase II polypeptide.

15. The method of claim 1, wherein said exogenous nucleic acid further comprises a nucleotide sequence encoding a screenable marker.

16. The method of claim 15, wherein said screenable marker is a GFP polypeptide or a β-glucuronidase polypeptide.

17. The method of claim 1, wherein said method further comprises regenerating at least one Miscanthus plant from said transformed callus.

18. The method of claim 17, wherein said regeneration step comprises culturing said transformed callus on a medium comprising MS basal salts, B5 vitamins, 2 mg/L BA, 0.1 mg/L NAA, 1% maltose, 50 mg/L L-glutamine, 2% sucrose, 7 g/L agar, 50 mg/L paromomycin, and 125 mg/L TIMENTIN®, at pH 5.8.

19. The method of claim 18, wherein said regeneration step comprises subsequently culturing said transformed callus on a medium comprising ½ MS basal salts, 0.2 mg/L glycine, 0.5 mg/L thiamine, 1.0 mg/L pyridoxine, 1.0 mg/L nicotinic acid, 100 mg/L myo-inositol, 1.5% sucrose, 0.5 mg/L NAA, 125 mg/L TIMENTIN®, and 2.5 g/L GELRITE®, at pH 5.8.

20. A transgenic Miscanthus plant, wherein said plant comprises an exogenous nucleic acid comprising a selectable marker and at least one T-DNA border.

21. The transgenic plant of claim 20, wherein said at least one T-DNA border is a right T-DNA border.

22. A method of transforming Miscanthus, said method comprising
(a) contacting an embryogenic Miscanthus callus with Agrobacterium comprising an exogenous nucleic acid, wherein said exogenous nucleic acid comprises a nucleic acid sequence encoding a selectable marker conferring resistance to a selection agent; and
(b) selecting for at least one transformed callus, derived from step (a), in a medium containing said selection agent, wherein said selection medium inhibits the growth of said Agrobacterium.

23. A method of transforming Miscanthus, said method comprising
(a) inducing an embryogenic callus from Miscanthus tissue on an induction medium;
(b) contacting said induced embryogenic callus with Agrobacterium comprising an exogenous nucleic acid, wherein said exogenous nucleic acid comprises a nucleic acid sequence encoding a selectable marker conferring resistance to a selection agent;
(c) selecting for at least one transformed callus, derived from step (b), in a medium containing said selection agent, wherein said selection medium inhibits the growth of said Agrobacterium; and
(d) regenerating at least one Miscanthus plant from said transformed callus.

24. A transgenic Miscanthus plant, wherein said plant comprises an exogenous nucleic acid comprising at least one T-DNA border incorporated into its genome.

25. A method of transforming Miscanthus, said method comprising
(a) contacting an embryogenic Miscanthus callus with Agrobacterium comprising an exogenous nucleic acid in a co-cultivation medium comprising from 125 μM to 400 μM acetosyringone, under conditions wherein said callus is infected by said Agrobacterium, wherein said exogenous nucleic acid comprises a nucleic acid sequence encoding a selectable marker conferring resistance to a selection agent; and
(b) selecting for at least one transformed callus, derived from step (a), in a medium containing said selection agent, wherein said selection medium inhibits the growth of said Agrobacterium.
